(19) United States
(12) Patent Application Publication
(10) Pub. No.: US 2026/0055470 A1
(43) Pub. Date: Feb. 26, 2026

(54) COMPOSITIONS AND METHODS FOR DETECTING OVARIAN CANCER

(71) Applicant: UNIVERSITY OF IOWA RESEARCH FOUNDATION, Iowa City, IA (US)

(72) Inventor: Jesus Gonzalez Bosquet, Iowa City, IA (US)

(73) Assignee: UNIVERSITY OF IOWA RESEARCH FOUNDATION, Iowa City, IA (US)

(21) Appl. No.: 19/254,263

(22) Filed: Jun. 30, 2025

Related U.S. Application Data
(60) Provisional application No. 63/666,507, filed on Jul. 1, 2024.

(51) Int. Cl. C12Q 1/6886

(52) U.S. Cl. CPC C12Q 1/6886; C12Q 2600/154; G01N 2800/50

(57) ABSTRACT

In certain embodiments, the present invention provides a panel of probes to detect biomarkers associated with ovarian cancer, wherein the probes hybridize to the biomarkers. Certain embodiments provide kits that comprise a panel of probes to detect biomarkers associated with ovarian cancer. In certain embodiments, the present invention provides a method of detecting the presence of biomarkers associated with an increased risk of ovarian cancer in a human subject. In certain embodiments, the present invention provides a method of treating a human subject for ovarian cancer.

### CROSS-REFERENCE TO RELATED APPLICATION

This application claims priority to U.S. Provisional Application No. 63/666,507 that was filed on Jul. 1, 2024. The entire content of the applications referenced above is hereby incorporated by reference herein.

### SEQUENCE LISTING

The instant application contains a Sequence Listing which has been submitted in XML format via Patent Center and is hereby incorporated by reference in its entirety. Said XML copy, created on Sep. 25, 2025, is named 17023291US1.xml and is 9,248 bytes in size.

### BACKGROUND

Molecular profiling of tumors obtained from individual patients improves the selection of personalized cancer treatment therapies, patient responses, detection of drug resistance, and monitoring of tumor relapse. Profiling tumors generally involves obtaining resected tumor samples by invasive surgeries. The limitations to such invasive procedures include difficulty in acquiring tumor samples for both tumor quantity and quality. Another drawback is that acquiring biopsy samples by invasive methods throughout treatment to monitor tumor response and relapse pose major challenges in tumor profiling. A further limitation to invasive sampling methods is the heterogeneity of resected tumor samples as a whole. Further, in the case of metastasis, where tumors have spread and constantly evolve both spatially and temporally in response to treatment over time, multiple biopsies may be required. These challenges make it difficult to obtain a holistic image of a tumor.

Recently, new non-invasive techniques are being developed to address these limitations, such as liquid biopsy (LB). Liquid biopsies consist of isolating tumor-derived entities like circulating tumor cells, circulating tumor DNA, tumor extracellular vesicles, etc., present in the body fluids of patients with cancer, followed by an analysis of genomic and proteomic data contained within them. Liquid biopsies methods permit continuous monitoring by repeated sampling. Further, LB provides enhanced sensitivity in diagnosis and ease of repeated sampling throughout treatment much more conveniently and non-invasively.

Various groups have attempted to increase the accuracy of pre-operative diagnosis of pelvic masses. Traditionally, the methods involved tumor markers, such as CA-125, HE-4, among others with or without the addition of ultrasound imaging characteristics and patient menopausal status. These methods offer sensitivities and specificities in the 70-80% range. More recently, groups have begun using machine learning and genomic information to create models that would better identify these models. To this point, however, they have not yet improved upon the tumor marker model. Accordingly, a minimally invasive model is needed that increases the accuracy of pre-operative diagnosis of pelvic masses.

### SUMMARY

One aspect provides a panel of probes that hybridize to nucleic acid biomarkers associated with ovarian cancer, the panel comprising at least seven probes selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:9.

In certain aspects, the panel comprises at least eight probes.

In certain aspects, the panel comprises at least nine probes.

In certain aspects, each probe comprises a unique label.

One aspect provides a kit comprising the panel of probes of any one of claims 1-4 and instructions for use in analyzing a biological sample.

In certain aspects, the biological sample is a liquid biopsy.

In certain aspects, the liquid biopsy is blood or a blood product.

One aspect provides a method of detecting the presence of nucleic acid biomarkers associated with an increased risk of ovarian cancer in a human subject, comprising: (i) contacting DNA from a biological sample containing cells from the subject with the panel of probes as described above to form hybridized biomarkers; (ii) detecting the hybridized biomarkers; (iii) determining the number of biomarker sequences detected; and (iv) indicating that the human subject has an increased risk of ovarian cancer.

In certain aspects, the biological sample is a liquid biopsy.

In certain aspects, the liquid biopsy is blood or a blood product.

In certain aspects, the biological sample is subdivided into individual subsamples, and a different single probe is applied to each subsample.

One aspect provides a method of treating a human subject for ovarian cancer, comprising: (i) contacting cfDNA from a liquid biopsy from the subject, with a panel of probes as described above to form hybridized biomarkers; (ii) detecting the hybridized biomarkers; (iii) determining the number of hybridized biomarkers detected; and (iv) indicating that the human subject has an increased risk of ovarian cancer, and (v) administering an appropriate treatment to the patient.

In certain aspects, the treatment comprises assessment of fallopian tubes and ovaries with imaging techniques or removal of tubes and ovaries. In certain aspects, the imaging techniques are CT scan, MRI and/or ultrasound.

In certain aspects, the panel comprises at least nine probes.

In certain aspects, the treatment comprises assessment of fallopian tubes and ovaries with imaging techniques or removal of fallopian tubes and ovaries.

One aspect provides a method of determining an increased risk or presence of ovarian cancer in a human subject comprising: (i) contacting cfDNA from a liquid biopsy from the subject, with a panel of probes as described above to form hybridized biomarkers;
(ii) detecting the hybridized biomarkers;
(iii) determining the number of hybridized biomarkers detected; and
(iv) indicating that the human subject has an increased risk of ovarian cancer.

### BRIEF DESCRIPTION OF DRAWINGS

The patent or application file contains at least one drawing executed in color. Copies of this patent or patent application publication with color drawings will be provided by the Office upon request and payment of the necessary fee.

FIGS. 1A-1F: DNA-methylation model to predict HGSC. MethylNet hyperparameter selection and prediction for HGSC: FIG. 1A. Preprocessing with normalization, selection of autosomal chromosomal markers, elimination of outliers. Training with 80% of samples, validating with 12.5%, 400 repetitions with most informative 300,000 DNA-methylation features. Representation of all samples with Uniform Manifold Approximation and Projection (UMAP), which is a dimension reduction technique that can be used for visualization of high-dimensional data by giving each datapoint a location in a three-dimensional map (in our sample: x, y and z). FIG. 1B. After MethylNet prediction analysis, 23,397 DNA-methylated features were selected for the best model to predict HGSC with a performance of 100% measured by the AUC. Variable reduction with lasso regression analysis: FIG. 1C. Heatmap of the 11, 167 significant DNA methylated probes (out of 23,397, p<0.05) after univariate analysis with ANOVA and cross-validation, repeated 10 times (caret R package). FIG. 1D. After lasso multivariate regression analysis (glmnet R package), there were 9 probes that predicted HGSC (upper axis) with a performance of 100%, measured by AUC (y axis). FIG. 1E. (SEQ ID NOs: 1-9) Table with the predictive probes of HGSC in the multivariate analysis: OR: odds Ratio of HGSC: <1, less risk of cancer, >1 more risk of cancer; also, chromosome (chr) position on the chromosome (position); direction of the transcription (strand); UCSC reference gene, where they are located in the gene (group), and what type of DNA methylation the probe is measuring. Chromosomal (chr) and genomic location (GRCh38 genomic location) of the methylation probes, including the strand of the DNA interrogated, their relationship with genome CpG islands based on the manufacturer annotation, if the probe is interrogating a known gene (and where is related to that gene-Group), and the actual probe sequence. FIG. 1F. Symbol, full name and description of the protein encoded by the gene interrogated by the methylated probe.

FIGS. 2A-2F. Validation and testing of prediction models of HGSC with DNA-methylation. FIGS. 2A-2B. External validation of prediction models in independent dataset GSE65820: External validation of the model that included 11,167 significant DNA methylated probes in the independent DNA methylation dataset (GSE65820) had an excellent performance of 98% (95% CI: 95%, 100%), measured by the AUC. FIG. 2B. The validation of the simplified lasso multivariate analysis including only the nine most informative probes, had a very good performance with an AUC of 84% (95% CI: 76%, 93%). Both validations were performed with the pROC R package. FIG. 2C. Training, validation and testing of the model using resulting probes from MethylNet prediction model of HGSC (N=23,397) performed in the UI dataset with ML analytics: the upper panel shows the testing confusion matrix representing the observed versus the predicted values; the lower panel represents the ROC graphic including models accounting for imbalanced samples: Train R: results of imbalanced (or re-sampling) training model; Test R: results of re-sampling testing model. FIG. 2D. Training, validation and testing using probes selected in the ANOVA univariate analysis with cross-validation (N=11,167) performed in the UI dataset with ML analytics: the upper panel shows the testing confusion matrix; the lower panel represents the ROC graphic including models accounting for imbalanced samples: Train R: results of imbalanced training model; Test R: results of re-sampling testing model. FIG. 2E. Training, validation and testing using all 9 probes found to be more informative in the lasso multivariate analysis performed in the UI dataset with ML analytics: the upper panel shows the testing confusion matrix; the lower panel represents the ROC graphic including models accounting for imbalanced samples. FIG. 2F. External validation of the UI model, performing training, validation and testing in the GSE65820 dataset with ML analytics using the most informative 9 probes in the lasso multivariate analysis: the upper panel shows the testing confusion matrix; lower panel represents the ROC graphic including: 1) models accounting for weights of the outcome: Train W: results of weighted training model; Test W: results of weighted testing model; 2) models accounting for imbalanced samples.

FIG. 3. Heatmap of the 9-probe models of HGSC prediction. UI dataset model: (left panel) Cluster of the methylation values of the 9 probes included in the UI model. Tube (or control) values are on the left. On the top there are clinical variables for this cohort: age, stage, grade and residual tumor after surgery. Rec: recurrence, Unk: unknown. The data matrix legend (on the right) shows the methylation percentage of the probes (beta, or β, value): 0=unmethylated, 1=100% methylated. GSE65820 external validation dataset model: (right panel) Cluster of the methylation values of the 9 probes included in the external validation model. Tube (or control) values are on the left.

### DETAILED DESCRIPTION

### Definitions

Unless otherwise indicated, the practice of the method and system disclosed herein involves conventional techniques and apparatus commonly used in molecular biology, microbiology, protein purification, protein engineering, protein and DNA sequencing, and recombinant DNA fields, which are within the skill of the art. Such techniques and apparatus are known to those of skill in the art and are described in numerous texts and reference works.

Numeric ranges are inclusive of the numbers defining the range. It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The headings provided herein are not intended to limit the disclosure.

Unless defined otherwise herein, all technical and scientific terms used herein have the same meaning as commonly understood by one of ordinary skill in the art. Various scientific dictionaries that include the terms included herein are well known and available to those in the art. Although any methods and materials similar or equivalent to those described herein find use in the practice or testing of the embodiments disclosed herein, some methods and materials are described.

The terms defined immediately below are more fully described by reference to the Specification as a whole. It is to be understood that this disclosure is not limited to the particular methodology, protocols, and reagents described, as these may vary, depending upon the context they are used by those of skill in the art. As used herein, the singular terms “a,” “an,” and “the” include the plural reference unless the context clearly indicates otherwise.

Unless otherwise indicated, nucleic acids are written left to right in 5′ to 3′ orientation and amino acid sequences are written left to right in amino to carboxy orientation, respectively.

The term “nucleic acid” refers to deoxyribonucleic acid (DNA) or ribonucleic acid (RNA) and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues.

The terms “polynucleotide”, “nucleic acid” and “nucleic acid fragment” are used interchangeably herein. These terms encompass nucleotides connected by phosphodiester linkages. A “polynucleotide” may be a ribonucleic acid (RNA) or deoxyribonucleic acid (DNA) polymer that is single- or double-stranded, that optionally contains synthetic, non-natural or altered nucleotide bases. A polynucleotide in the form of a polymer of DNA may comprise one or more segments of cDNA, genomic DNA, synthetic DNA, or mixtures thereof. Nucleotide bases are indicated herein by a single letter code: adenine (A), guanine (G), thymine (T), cytosine (C), inosine (I) and uracil (U). Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

The invention encompasses isolated or substantially purified nucleic acid compositions. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. Fragments and variants of the disclosed nucleotide sequences are also encompassed by the present invention. By “fragment” or “portion” is meant a full length or less than full length of the nucleotide sequence.

A gene is a locus (or region) of DNA which is made up of nucleotides and is the molecular unit of heredity.

Genes can acquire mutations in their sequence, leading to different variants, known as alleles, in the population. These alleles encode slightly different versions of a protein, which cause different phenotype traits.

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have at least 40, 50, 60, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

The term “test sample” herein refers to a sample, typically derived from a biological fluid, cell, tissue, organ, or organism, comprising a nucleic acid or a mixture of nucleic acids comprising at least one nucleic acid sequence that is to be screened. In certain embodiments the sample comprises at least one nucleic acid sequence whose copy number is suspected of having undergone variation. Such samples include, but are not limited to sputum/oral fluid, amniotic fluid, blood, a blood fraction, or fine needle biopsy samples (e.g., surgical biopsy, fine needle biopsy, etc.), urine, peritoneal fluid, pleural fluid, and the like. Although the sample is often taken from a human subject (e.g., patient), the assays can be used in samples from any mammal, including, but not limited to dogs, cats, horses, goats, sheep, cattle, pigs, etc. The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. If such methods of pretreatment are employed with respect to the sample, such pretreatment methods are typically such that the nucleic acid(s) of interest remain in the test sample, sometimes at a concentration proportional to that in an untreated test sample (e.g., namely, a sample that is not subjected to any such pretreatment method(s)). Such “treated” or “processed” samples are still considered to be biological “test” samples with respect to the methods described herein.

The term “sequence of interest” or “nucleic acid sequence of interest” or “target sequence” or “biomarker” herein refers to a nucleic acid sequence that is associated with a difference in sequence representation between healthy and diseased individuals.

The terms “threshold value” and “qualified threshold value” herein refer to any number that is used as a cutoff to characterize a sample such as a test sample containing a nucleic acid from an organism suspected of having a medical condition. The threshold may be compared to a parameter value to determine whether a sample giving rise to such parameter value suggests that the organism has the medical condition. In certain embodiments, a qualified threshold value is calculated using a qualifying data set and serves as a limit of diagnosis of an ovarian cancer target biomarker. If a threshold is exceeded by results obtained from methods disclosed herein, a subject can be diagnosed with an ovarian cancer (e.g., having target biomarkers).

The term “clinically-relevant sequence” or “clinically-relevant biomarker” herein refers to a nucleic acid sequence that is known or is suspected to be associated or implicated with a genetic or disease condition. Determining the absence or presence of a clinically-relevant sequence can be useful in determining a diagnosis or confirming a diagnosis of a medical condition, or providing a prognosis for the development of a disease.

The term “derived” when used in the context of a nucleic acid or a mixture of nucleic acids, herein refers to the means whereby the nucleic acid(s) are obtained from the source from which they originate. For example, in one embodiment, a mixture of nucleic acids that is derived from two different genomes means that the nucleic acids, e.g., cfDNA, were naturally released by cells through naturally occurring processes such as necrosis or apoptosis. In another embodiment, a mixture of nucleic acids that is derived from two different genomes means that the nucleic acids were extracted from two different types of cells from a subject.

The term “based on” when used in the context of obtaining a specific quantitative value, herein refers to using another quantity as input to calculate the specific quantitative value as an output.

The term “biological fluid” herein refers to a liquid taken from a biological source and includes, for example, blood, serum, plasma, sputum, lavage fluid, cerebrospinal fluid, urine, semen, sweat, tears, saliva, and the like. As used herein, the terms “blood,” “plasma” and “serum” expressly encompass fractions or processed portions thereof. Similarly, where a sample is taken from a biopsy, swab, smear, etc., the “sample” expressly encompasses a processed fraction or portion derived from the biopsy, swab, smear, etc.

The term “subject” herein refers to a human subject as well as a non-human subject such as a mammal, an invertebrate, a vertebrate, a fungus, a yeast, a bacterium, and a virus. Although the examples herein concern humans and the language is primarily directed to human concerns, the concepts disclosed herein are applicable to genomes from any plant or animal, and are useful in the fields of veterinary medicine, animal sciences, research laboratories and such.

The term “condition” herein refers to “medical condition” as a broad term that includes all diseases and disorders, but can include injuries and normal health situations, such as pregnancy, that might affect a person's health, benefit from medical assistance, or have implications for medical treatments.

The term “sensitivity” as used herein refers to the probability that a test result will be positive when the condition of interest is present. It may be calculated as the number of true positives divided by the sum of true positives and false negatives.

The term “specificity” as used herein refers to the probability that a test result will be negative when the condition of interest is absent. It may be calculated as the number of true negatives divided by the sum of true negatives and false positives.

The term “enrich” herein refers to the process of amplifying polymorphic target nucleic acids contained in a portion of a biological sample and combining the amplified product with the remainder of the biological sample from which the portion was removed. For example, the remainder of the biological sample can be the original biological sample.

The methods described herein can be used for the detection, diagnosis, targeting, and treatment of a subject having cancer, in particular, ovarian cancer.

In certain embodiments, patients have or are suspected of having ovarian cancer.

Examples of locations where sample collection may be performed include health practitioners' offices, clinics, patients' homes (where a sample collection tool or kit is provided), and mobile health care vehicles. Examples of locations where sample processing may be performed include health practitioners' offices, clinics, patients' homes (where a sample processing apparatus or kit is provided), mobile health care vehicles, and facilities of biomarker analysis providers.

In certain embodiments, the diagnosis is generated at the same location as the analyzing operation. In other embodiments, it is performed at a different location. In some examples, reporting the diagnosis is performed at the location where the sample was taken, although this need not be the case. Examples of locations where the diagnosis can be generated or reported and/or where developing a plan is performed include health practitioners' offices, clinics, internet sites accessible by computers, and handheld devices such as cell phones, tablets, smart phones, etc. having a wired or wireless connection to a network. Examples of locations where counseling is performed include health practitioners' offices, clinics, internet sites accessible by computers, handheld devices, etc.

In some embodiments, the sample collection and sample processing are performed at a first location and the analyzing and deriving operation is performed at a second location.

### Probes, Diagnostic Panels and Kits

In certain embodiments, the present invention provides a diagnostic panel of probes that hybridize to nucleic acid. In certain embodiments the nucleic acid is cfDNA.

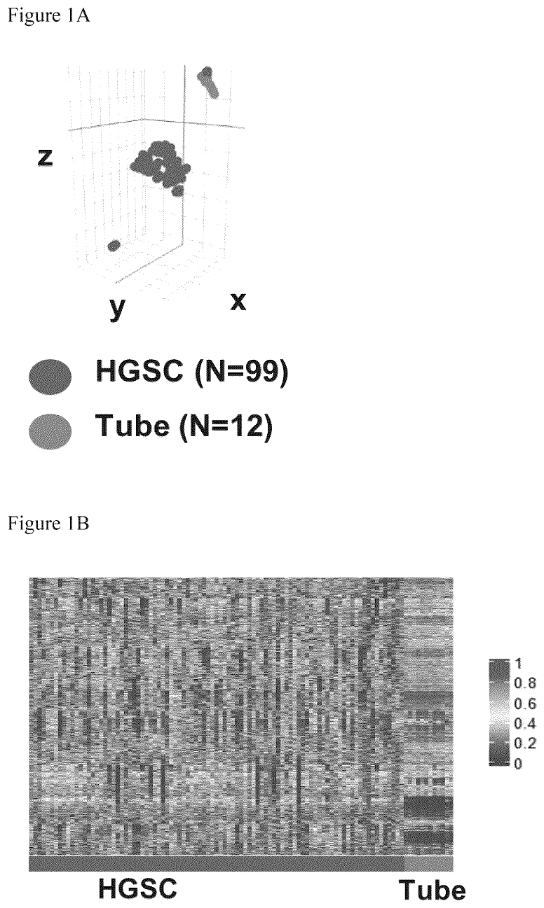

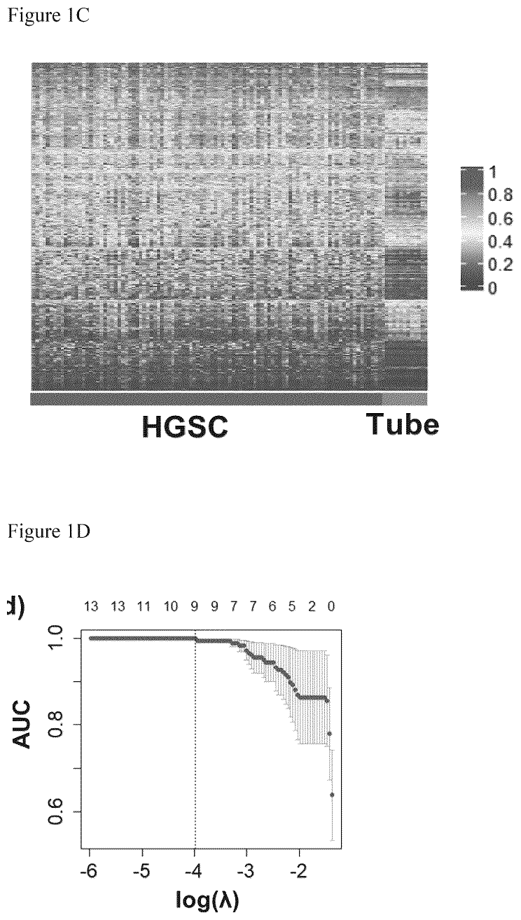

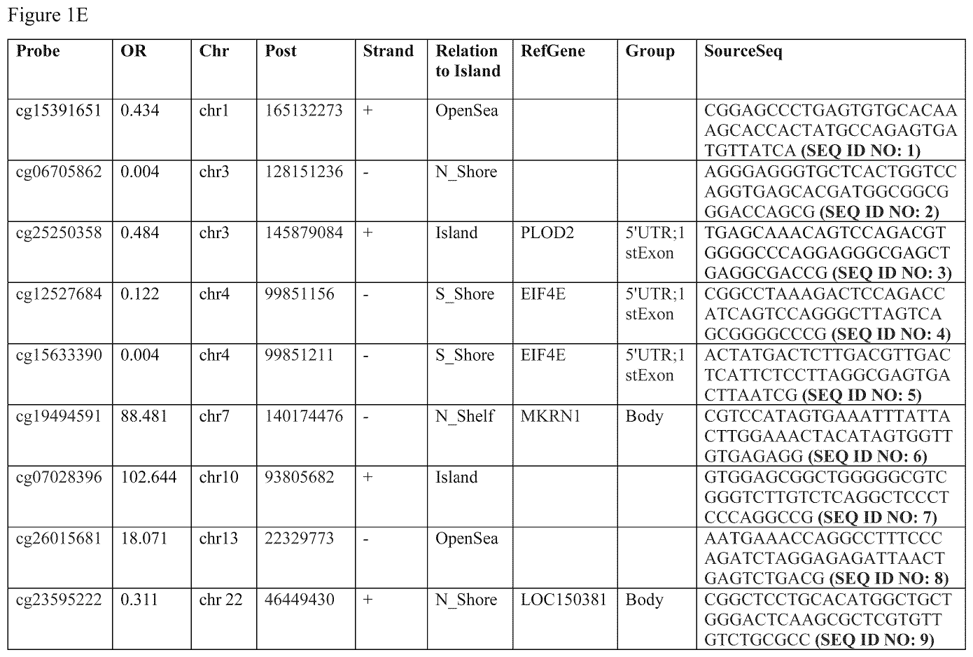

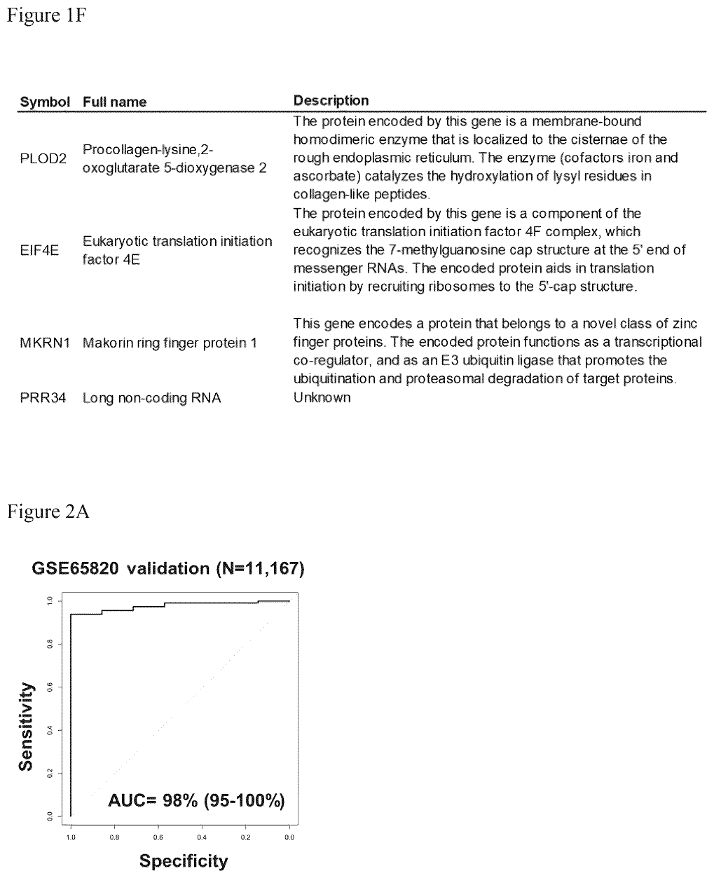

In certain embodiments, the panel comprises a probe selected from the group consisting of the probes provided in FIG. 1E:

(SEQ ID NO: 1)
CGGAGCCCTGAGTGTGCACAAAGCACCACTATGCCAGAGTGATGTTATCA
(SEQ ID NO: 2)
AGGGAGGGTGCTCACTGGTCCAGGTGAGCACGATGGCGGCGGGACCAGCG
(SEQ ID NO: 3)
TGAGCAAACAGTCCAGACGTGGGGCCCAGGAGGGCGAGCTGAGGCGACCG
(SEQ ID NO: 4)
CGGCCTAAAGACTCCAGACCATCAGTCCAGGGCTTAGTCAGCGGGGCCCG
(SEQ ID NO: 5)
ACTATGACTCTTGACGTTGACTCATTCTCCTTAGGCGAGTGACTTAATCG
(SEQ ID NO: 6)
CGTCCATAGTGAAATTTATTACTTGGAAACTACATAGTGGTTGTGAGAGG
(SEQ ID NO: 7)
GTGGAGCGGCTGGGGGCGTCGGGTCTTGTCTCAGGCTCCCTCCCAGGCCG
(SEQ ID NO: 8)
AATGAAACCAGGCCTTTCCCAGATCTAGGAGAGATTAACTGAGTCTGACG
(SEQ ID NO: 9)
CGGCTCCTGCACATGGCTGCTGGGACTCAAGCGCTCGTGTTGTCTGCGCC

In certain embodiments, the panel comprises at least one, two, three, four, five, six, seven, eight, or nine probes.

In certain embodiments, a probe is operably linked to a detection moiety.

In certain embodiments, each probe in the panel of probes is methylated.

In certain embodiments, a probe is methylated.

In certain embodiments, each probe in the panel of probes is operably linked to a genotyping microchip.

One aspect provides a kit comprising a collection of probes, wherein the collection comprises a panel of probes, and instructions for use in analyzing a biological sample. In certain embodiments, the panel comprises at least 2 probes specific for ovarian cancer. In certain embodiments, the kit comprises a panel containing at least one, two, three, four, five, six, seven, eight, or nine probes.

### Oligonucleotide Probes

The method of the present invention is useful for detecting the presence of biomarkers associated with ovarian cancer. The first step of the process involves contacting a physiological sample obtained from a patient, which sample contains nucleic acid, with an oligonucleotide probe to form a hybridized DNA. The oligonucleotide probes that are useful in the methods of the present invention are those listed in FIG. 1E.

Any oligonucleotide backbone may be employed, including DNA, RNA (although RNA is less preferred than DNA), modified sugars such as carbocycles, and sugars containing 2′ substitutions such as fluoro and methoxy. The oligonucleotides may be oligonucleotides wherein at least one, or all, of the inter-nucleotide bridging phosphate residues are modified phosphates, such as methyl phosphonates, methyl phosphonotlioates, phosphoroinorpholidates, phosphoropiperazidates and phosplioramidates.

In certain embodiments, the probes are labeled. Nucleic acids are readily labeled with tags that facilitate detection or purification. A variety of enzymatic or chemical methods are known in the art to generate nucleic acids labeled with radioactive phosphates, fluorophores, or nucleotides modified with biotin or digoxygenin, for example.

In certain embodiments, the nucleic acid probes are labeled at their 5′-end, their 3′-end, or throughout the molecule. In certain embodiments, labels are distributed throughout the nucleic acid, through techniques such as nick translation, random priming, by PCR or in vitro transcription using labeled dNTPs or NTPs.

### Ovarian Cancer-Specific Biomarkers and System of Detection

Preoperative diagnosis of ovarian cancer remains challenging. More accurate detection of ovarian cancer at an earlier stage would reduce the number of unnecessary surgeries on benign pelvic masses. Currently, tumor markers and imaging characteristics offer only limited sensitivity and specificity, and surgical excision is the only definitive way to achieve diagnosis. The present inventors have developed systems to more accurately detect the presence of ovarian cancer in a patient at an earlier stage, which allows for improved patient outcomes.

In certain aspects of the present invention, a system has been developed that detects biomarkers associated with ovarian cancer. The system successfully makes this detection with about 70% to 100% accuracy. In certain aspects, the system successfully makes this distinction with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or to 100% accuracy.

Briefly, a biological sample is taken from a patient, and the nucleic acid present in the sample is analyzed using the probes listed in FIG. 1E to generate a “patient signature.” If at least seven or more diagnostic model biomarkers are present in the patient signature, a high-certainty diagnosis is made that the patient has ovarian cancer. In certain aspects, at least nine diagnostic biomarkers are present. Thus, a high-certainty diagnosis is made with a simple, peripheral blood draw. Based on the diagnosis, appropriate treatment is commenced. This method is useful for diagnosis in patients with ovarian cancer, for screening high-risk populations, or in surveillance for ovarian cancer recurrence.

### Biological Samples

Since blood contacts most tumors, liquid biopsies (LBs) mostly involve blood sampling, although other body fluids like mucosa, pleural effusions, urine, and cerebrospinal fluid (CSF) are also analyzed. In certain embodiments, a biological sample is obtained from a patient. In certain embodiments, the sample is a liquid biopsy, such as a blood or plasma sample. In certain embodiments, the sample is mucosa, pleural effusions, urine, and cerebrospinal fluid (CSF).

In certain embodiments, the sample contains circulating tumor cells (CTCs) that are shed by both primary and metastatic tumors, circulating tumor DNA (ctDNA), tumor derived extracellular vesicles (EVs) that are membrane-bound subcellular moieties composed of nucleic acids/proteins, tumor educated platelets (TEPs), and circulating cell-free DNA (cfDNA).

### Circulating Tumor DNA in Liquid Biopsies

In certain embodiments, circulating tumor DNA (ctDNA) is present in the liquid biopsy. Over time, fragments of DNA from the tumor cells can enter a patient's bloodstream, and this DNA is called circulating tumor DNA (ctDNA). This ctDNA can be from dying tumor cells or as the cancer cells turnover. Circulating tumor DNA (ctDNA) is single- or double-stranded DNA released by the tumor cells into the blood and it thus harbors the mutations of the original tumor. Circulating tumor DNA (ctDNA) is distinguishable from cell-free DNA (cfDNA), in that DNA fragments shed from non-tumor cells are cfDNA, whereas DNA fragments shed from tumors are ctDNA. ctDNA accounts for about 0.1-10% of the total circulating cell-free DNA (cfDNA). ctDNA levels in plasma, however, can vary depending on tumor load, tumor stage, and therapeutic response. Recent studies have shown that ctDNA differs in length from the cfDNA, with reports indicating ctDNA fractions in patients with cancer to be 20-50 base pairs, which is generally shorter than cfDNA.

### Non-Blood Liquid

In addition to circulatory fluids like plasma or serum, other body fluids such as saliva and urine can be used as liquid biopsies. Saliva offers practical advantages with regard to ease of access, non-invasiveness, and cost effectiveness in sampling, even more so than plasma or serum. Novel electrochemical sensor-based technologies like an electric field-induced release and measurement (EFIRM) have been shown to detect EGFR mutations (tyrosine kinase domain) from bodily fluids like saliva in patients. Similar EFIRM based technologies have been used in developing salivary biomarkers.

The completely non-invasive nature of urine sampling, relative to tissue or even blood, makes it a quite useful candidate in LBs, particularly in cases where repeated sampling is required to monitor tumor progression and therapeutic outcomes.

### Hybridization Methodology

The DNA (or nucleic acid) sample may be contacted with the oligonucleotide probe in any suitable manner known to those skilled in the art. For example, the DNA sample may be solubilized in solution and contacted with the oligonucleotide probe by solubilizing the oligonucleotide probe in solution with the DNA sample under conditions that permit hybridization. Suitable conditions are well known to those skilled in the art. Alternatively, the DNA sample may be solubilized in solution with the oligonucleotide probe immobilized on a solid support, whereby the DNA sample may be contacted with the oligonucleotide probe by immersing the solid support having the oligonucleotide probe immobilized thereon in the solution containing the DNA sample.

### Methods of Detecting Biomarkers Specific for Ovarian Cancer

In certain embodiments, a blood sample is taken and DNA (e.g., ctDNA) is extract from the sample. Then, genomic regions are amplified around the nine targets via standard PCR on a thermal cycler using primers specific for those regions. Next, the products are hybridized to a custom chip with reference and alternative alleles of each of these loci. The resulting genotypes are analyzed for risk of ovarian cancer. If nine biomarker targets are present, then the patient has a high risk of presenting ovarian cancer, if fewer than seven targets are present, then the risk is low.

### Methods of Treating a Patient Identified as Having Biomarkers Specific for Ovarian Cancer

Once the patient signature is determined for the DNA (e.g., ctDNA) found in the sample and compared to the ovarian cancer “diagnostic panel” appropriate treatment is commenced. If the final risk of ovarian cancer is high by the “diagnostic panel” further study is recommend either by radiology or surgery depending on the surgical risk of the patient. If the surgical risk is minimal and the ovarian risk is above the cut-off (33%), a conversation would be initiated with the patient about potential removal of tubes and ovaries for ovarian cancer risk reduction.

The invention will now be illustrated by the following non-limiting Examples.

### Example 1

### Using Genomic Variation to Diagnose Ovarian Cancer

### Abstract

The majority of patients with epithelial ovarian cancer (EOC) continue to be diagnosed at an advanced stage despite great advances in this disease treatment. To impact overall survival, better methods of EOC are needed for early diagnosis. The following study was performed to predict high-grade serous cancer (HGSC) using artificial intelligence (AI) methodology and methylated DNA from surgical specimens. Initial prediction models with MethylNet were accurate but complex (AUC=100%). These models were optimized by selecting the most informative probes with univariate ANOVA analyses first, and then multivariate lasso regression modelling. This step-wise approach resulted in nine methylated probes predicting HGSC with an AUC of 100%. These models were validated with different analytics and with an independent DNA-methylation experiment with excellent performances.

### INTRODUCTION

Despite notable advances in treatment, patients with epithelial ovarian cancer (EOC) continue to have a high case-fatality rate. This is due in large part to most patients presenting with advanced, symptomatic disease. Conventional methods of diagnosis (imaging, CA125) lack the desired sensitivity and/or specificity to be effective and/or accurate for early diagnosis. Therefore, there is a critical need to develop new strategies with this goal.

One of the most exciting novel methods for early diagnosis is detection of cancer genomic material in blood with cell-free DNA (cfDNA) analysis, or “liquid biopsy.” To be successful in this endeavor though, adequate markers, analytics and procedures have to be developed. DNA-methylation is an effective screening tool for colon cancer and also has demonstrated potential for ovarian cancer detection. Additionally, artificial intelligence (AI) could be applied to improve the performance of predicting diagnostic tools.

The ultimate goal of the present study was to develop a method that could be used in early detection of ovarian cancer with cfDNA methodology. The primary aim was to build and validate a prediction model of high-grade serous ovarian cancer (HGSC—the most common EOC) using deep machine learning (AI) and DNA methylation data.

### Methods

This study included samples from patients with HGSC (N=99) and normal fallopian tube samples as controls (N=12). (Table 1, FIG. 1A).

TABLE 1
Characteristics of ovarian cancer patients included for analysis.

| Characteristic | |
|---|---|
| Age (mean) | 60 |
| Stage | |
| I-II | 4 |
| III | 62 |
| IV | 26 |
| Recurrent | 6 |
| unk | 1 |
| Preop CA125 (mean) | 2510 |
| Grade | |
| 1 | 3 |
| 2 | 21 |
| 3 | 64 |
| unk | 11 |
| Residual disease | |
| Microscopic | 19 |
| <1 | 43 |
| >1 | 36 |
| >2 | 37 |
| unk | 1 |

Age and preoperative CA125 levels are represented by their means. The rest of characteristics are counts. Unk: unknown.

Tissue samples and clinical outcome data were obtained from the Gynecologic Oncology Bank (IRB, ID #200209010) of the University of Iowa (UI). Clinical and pathological data were collected from the electronic medical record.

An Illumina Infinium MethylationEPIC BeadChip Array® was used to determine more than 850,000 DNA methylation features. Previously known methods of patient selection, DNA isolation, bisulfite conversion, and hybridization to the chip were used. Reyes, H. D. et al. Differential DNA methylation in high-grade serous ovarian cancer (HGSOC) is associated with tumor behavior. Scientific reports 9, 17996, doi: 10.1038/s41598-019-54401-w (2019). This Infinium array has more than 850,000 methylation datapoints (probes). To create a valid and effective model for a clinical setting extracted the most informative probes.

The initial variable reduction was performed with an open-source tool, MethylNet, which has been previously tested in TCGA (the Cancer genome Atlas) datasets successfully. Rather than introducing all variables resulting from the MethyNet analysis directly in the prediction lasso model, we reduce the number of variables introducing only features that were different between both groups in a univariate analysis with ANOVA with 10-fold cross-validation and 10 replicates for each fold. The goal was to decrease the complexity and background noise in the lasso multivariate prediction model so it can be more easily validated. Then, resulting models were optimized for clinical use using univariate and multivariate lasso regression modelling with k-fold cross-validation (caret and glmnet R packages). Performance of models were measured with the area under the curve (AUC) and their 95% confidence interval (CI).

The resulting models were validated in an independent database also available at the GEO repository, database GSE65820 and includes also fallopian tubes (N=7) and HGSC samples (N=114) and all the most informative probes resulting from the analysis. Classical learning statistical methods like pROC (R package) were used for external validation of these models. Additionally, an independent machine learning (ML) analytic platform, TensorFlow, was used for re-training, validation and testing of these prediction models in the UI dataset and in the independent GSE65820 database. 20% of the samples were used for validation and left out another 20% of the samples for testing prediction models. The analysis was performed in a Jupyter notebook with a Keras application programming interface (API). This notebook is a modification of the TensorFlow core tutorial ‘Classification of imbalanced data’.

### Results

The initial variable selection with MethylNet produced a model with 23,397 informative probes and a performance of 100% measured by the AUC (FIG. 1B). A model with such a number of variables is impractical. Therefore, multiple ANOVA univariate analysis was used to select those more informative (11,167 probes at a p-value<0.05) to be included in the multivariate analysis with lasso regression (FIG. 1B). The resulting lasso model comprised 9 informative probes (FIG. 1E) and had an AUC of 100% (FIG. 1D) and interrogated some known genes (FIG. 1F).

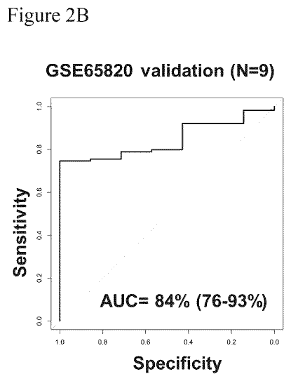

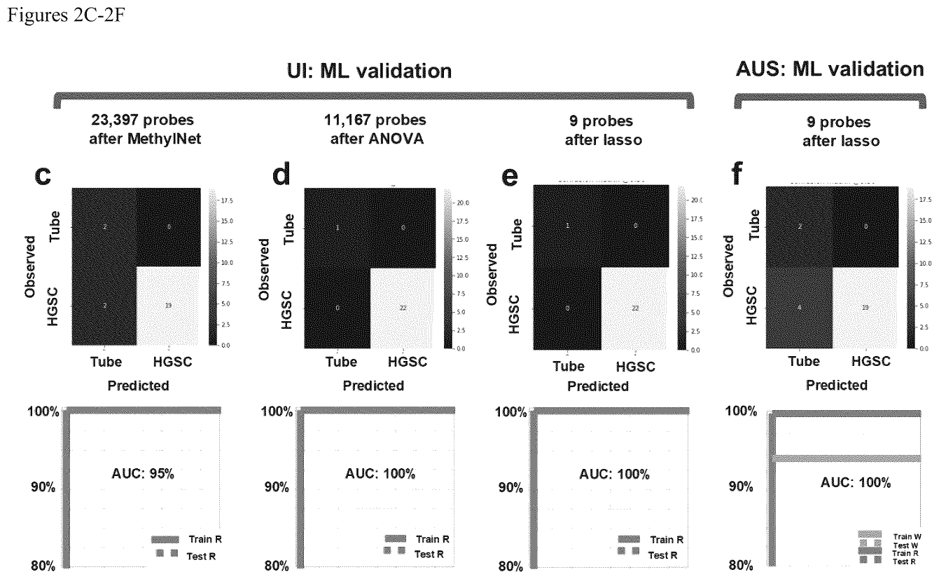

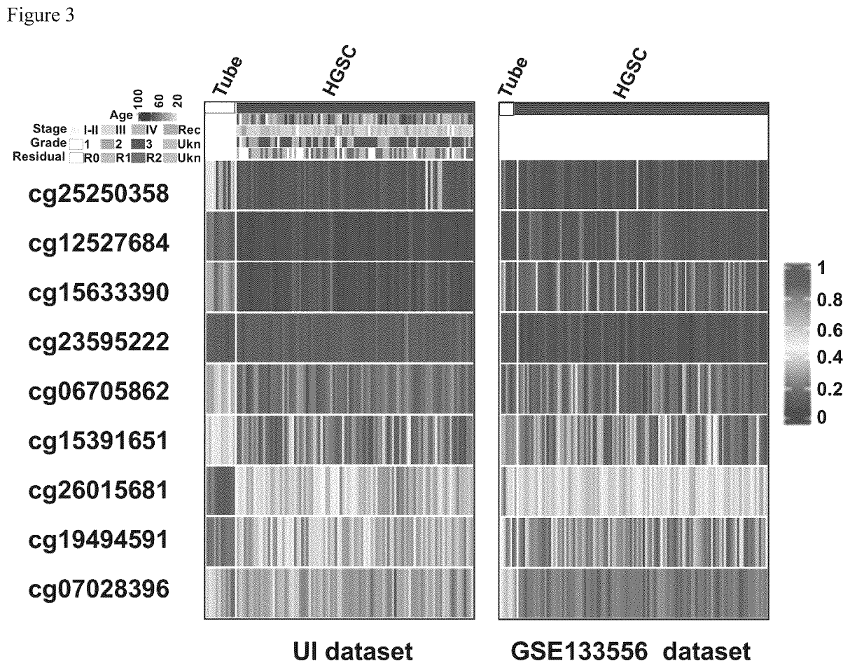

External validation of prediction models of HGSC created in the UI set and applied to GSE65820 dataset had very good performances, with AUC of 98% (95% CI: 95-100%) for the model with 11,167 probes after the ANOVA analysis, and with an AUC of 84% (CI: 76-93%) for the model with nine probes after the lasso regression (FIG. 2A). Training, validation and testing of these models in a ML platform also had excellent performances as detailed in FIG. 2B. Similarities between the data structure of the 9-probe model from UI data and the external validation dataset, GSE65820, are detailed in FIG. 3 with a heatmap.

### Discussion

A prediction model for HGSC with the step-wise AI methodology and using methylated DNA probes. This model is accurate and robust across independent datasets, and superior to some models reported with clinical and biological data. External validation also had a high performance, with an AUC of 84 (95% CI of 76-93%), based on an objective evaluation of prediction models' external validation, which includes: obtaining a suitable dataset, making outcome predictions, evaluating predictive performance, assessing clinical usefulness, and clearly reporting findings.

MethylNet was useful to reduce the number of variables from over 850k included in the Infinium MethylationEPIC BeadChip Array (Illumina, Inc., San Diego, CA) to over 23K. While this reduction in complexity was clearly advantageous there is a ‘black box’ effect due to the limited information about how some of the variables were selected by this deep-learning tool. This effect was mitigated, though, because the downstream analyses to select the most informative probes were performed with well know analytics and methods.

Previous studies have demonstrated that methylated DNA extracted from tumors and analyzed with methylation arrays, like the one used in the present study (Infinium MethylationEPIC BeadChip Array) could be used to identify classifiers for prediction models, latter to be validated in cfDNA from blood. Furthermore, these methylation arrays have been used successfully to identify methylation probes in cfDNA specimens in diverse cancer models with high accuracy.

Strengths of the present study include the use of a single institution biobank that is well annotated clinically. A homogeneous phenotype (HGSC) in a single population is advantageous when measuring accuracy of prediction models. However, it could detract from the generalizability of the model. Thus, the model was validated in an independent database, from a different geographical location (Australia) but with similar patient ancestry (western Europeans). It was decided to perform training and validating in UI dataset, and external validation in the GSE133556 set, because the latter was more imbalanced (higher case/control ratio, with only 7 controls) and controls in the UI set were well defined clinically (no personal or familiar history of ovarian cancer) More imbalanced data with uncertain control phenotypes could lead to unwanted biases. These models are applicable to populations with similar backgrounds (ancestry).

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A panel of probes that hybridize to nucleic acid biomarkers associated with ovarian cancer, the panel comprising at least seven probes selected from the group consisting of SEQ ID NO:1, SEQ ID NO:2, SEQ ID NO:3, SEQ ID NO:4, SEQ ID NO:5, SEQ ID NO: 6, SEQ ID NO:7, SEQ ID NO:8, and SEQ ID NO:9.

2. The panel of probes of claim 1, wherein the panel comprises at least eight probes.

3. The panel of probes of claim 1, wherein the panel comprises at least nine probes.

4. The panel of probes of claim 1, wherein each probe comprises a unique label.

5. A kit comprising the panel of probes of claim 1 and instructions for use in analyzing a biological sample.

6. The kit of claim 5, wherein the biological sample is a liquid biopsy.

7. The kit of claim 5, wherein the liquid biopsy is blood or a blood product.

8. A method of detecting the presence of nucleic acid biomarkers associated with an increased risk of ovarian cancer in a human subject, comprising:
(i) contacting DNA from a biological sample containing cells from the subject with the panel of probes of claim 1 to form hybridized biomarkers;
(ii) detecting the hybridized biomarkers;
(iii) determining the number of hybridized biomarkers detected; and
(iv) indicating that the human subject has an increased risk of ovarian cancer.

9. The method of claim 8, wherein the biological sample is a liquid biopsy.

10. The method of claim 9, wherein the liquid biopsy is blood or a blood product.

11. The method of claim 8, wherein the biological sample is subdivided into individual subsamples, and a different single probe is applied to each subsample.

12. A method of treating a human subject for ovarian cancer, comprising:
(i) contacting ctDNA from a liquid biopsy from the subject, with a panel of probes of claim 1 to form hybridized biomarkers;
(ii) detecting the hybridized biomarkers;
(iii) determining the number of hybridized biomarkers detected; and
(iv) indicating that the human subject has an increased risk of ovarian cancer, and
(v) administering an appropriate treatment to the patient.

13. The method of claim 12, wherein the panel comprises at least nine probes.

14. The method of claim 12, wherein the treatment comprises assessment of fallopian tubes and ovaries with imaging techniques or removal of fallopian tubes and ovaries.

15. The method of claim 14, wherein the treatment comprises an assessment of fallopian tubes and ovaries with imaging techniques or removal of tubes and ovaries. In certain aspects, the imaging techniques are CT scan, MRI and/or ultrasound.

16. A method of determining an increased risk or presence of ovarian cancer in a human subject comprising:
(i) contacting ctDNA from a liquid biopsy from the subject, with a panel of probes of claim 1 to form hybridized biomarkers;
(ii) detecting the hybridized biomarkers;
(iii) determining the number of hybridized biomarkers detected; and
(iv) indicating that the human subject has an increased risk of ovarian cancer. that the human subject has an increased risk of ovarian cancer.


SEQUENCE LISTING (9 sequences)

SEQ ID NO: 1 - DNA, 50 bp, synthetic construct
  source 1..50: mol_type = other DNA
cggagccctgagtgtgcacaaagcaccactatgccagagtgatgttatca

SEQ ID NO: 2 - DNA, 50 bp, synthetic construct
  source 1..50: mol_type = other DNA
agggagggtgctcactggtccaggtgagcacgatggcggcgggaccagcg

SEQ ID NO: 3 - DNA, 50 bp, synthetic construct
  source 1..50: mol_type = other DNA
tgagcaaacagtccagacgtggggcccaggagggcgagctgaggcgaccg

SEQ ID NO: 4 - DNA, 50 bp, synthetic construct
  source 1..50: mol_type = other DNA
cggcctaaagactccagaccatcagtccagggcttagtcagcggggcccg

SEQ ID NO: 5 - DNA, 50 bp, synthetic construct
  source 1..50: mol_type = other DNA
actatgactcttgacgttgactcattctccttaggcgagtgacttaatcg

SEQ ID NO: 6 - DNA, 50 bp, synthetic construct
  source 1..50: mol_type = other DNA
cgtccatagtgaaatttattacttggaaactacatagtggttgtgagagg

SEQ ID NO: 7 - DNA, 50 bp, synthetic construct
  source 1..50: mol_type = other DNA
gtggagcggctgggggcgtcgggtcttgtctcaggctccctcccaggccg

SEQ ID NO: 8 - DNA, 50 bp, synthetic construct
  source 1..50: mol_type = other DNA
aatgaaaccaggcctttcccagatctaggagagattaactgagtctgacg

SEQ ID NO: 9 - DNA, 50 bp, synthetic construct
  source 1..50: mol_type = other DNA
cggctcctgcacatggctgctgggactcaagcgctcgtgttgtctgcgcc